SEQUENCE LISTING (24 sequences)

SEQ ID NO: 1 - DNA, 25 bp, Homo sapiens
gagaacaatgagaaccttcaggaga

SEQ ID NO: 2 - DNA, 23 bp, Homo sapiens
ttctggcgccggttacagaacca

SEQ ID NO: 3 - DNA, 21 bp, Homo sapiens
ggcgtgcagtacaactacgtg

SEQ ID NO: 4 - DNA, 21 bp, Homo sapiens
agtcaatgaggtcaggccggt

SEQ ID NO: 5 - DNA, 22 bp, Homo sapiens
gaaccagaggggagagacagag

SEQ ID NO: 6 - DNA, 22 bp, Homo sapiens
ccctcagcttgctttttaggag

SEQ ID NO: 7 - DNA, 21 bp, Homo sapiens
gcgccaactccaacgtgttct

SEQ ID NO: 8 - DNA, 21 bp, Homo sapiens
gtgatgatgtgcaccaggttc

SEQ ID NO: 9 - DNA, 21 bp, Homo sapiens
aggaggccttcactatcatgg

SEQ ID NO: 10 - DNA, 21 bp, Homo sapiens
gtgatgatgtgcaccaggttc

SEQ ID NO: 11 - DNA, 21 bp, Homo sapiens
gaggagaatggccagcaggaa

SEQ ID NO: 12 - DNA, 21 bp, Homo sapiens
gcgaacatctgctccacctca

SEQ ID NO: 13 - DNA, 20 bp, Homo sapiens
acagctgccaaggctaccta

SEQ ID NO: 14 - DNA, 20 bp, Homo sapiens
gcttttgacgtgcttgttga

SEQ ID NO: 15 - DNA, 25 bp, Homo sapiens
ctggacaagaaccagcgacagtgcg

SEQ ID NO: 16 - DNA, 26 bp, Homo sapiens
atcacgatcaggagggccacataggg

SEQ ID NO: 17 - DNA, 25 bp, Homo sapiens
ctggacaagaaccagcgacagtgcg

SEQ ID NO: 18 - DNA, 26 bp, Homo sapiens
atcacgatcaggagggccacataggg

SEQ ID NO: 19 - DNA, 20 bp, Homo sapiens
ttcttggctcggggtttgcc

SEQ ID NO: 20 - DNA, 20 bp, Homo sapiens
tgttgctgccgcgatccttg

SEQ ID NO: 21 - DNA, 21 bp, Homo sapiens
cctgaaccctaaggccaaccg

SEQ ID NO: 22 - DNA, 21 bp, Homo sapiens
gctcatagctcttctccaggg

SEQ ID NO: 23 - DNA, 20 bp, Homo sapiens
tggctttgcccctctcacca

SEQ ID NO: 24 - DNA, 20 bp, Homo sapiens
cggcggaacatggcagtgaa

(19) United States
(12) Patent Application Publication
(10) Pub. No.: US 2007/0161107 A1
(43) Pub. Date: Jul. 12, 2007

(54) Differentiation of human embryonic stem cells to cardiomyocytes

(76) Inventors: Christine Lindsay Mummery, Bilthoven (NL); Petrus Adrianus Frederik Maria Doevendans, Utrecht (NL); Leon Gerardus Joseph Tertoolen, Utrecht (NL)

Correspondence Address: SCULLY, SCOTT, MURPHY & PRESSER, P.C., 400 GARDEN CITY PLAZA, SUITE 300, GARDEN CITY, NY, 11530, US

(21) Appl. No.: 10/548,501

(22) PCT Filed: Mar. 11, 2004
(86) PCT No.: PCT/AU04/00302

§ 371 Date: Sep. 11, 2006

(30) Foreign Application Priority Data
Mar. 11, 2003 (AU) 2003901099

(51) Int. Cl. C12N 5/08

(52) U.S. Cl. 435/366; 435/370

(57) ABSTRACT

A method for inducing cardiomyocyte differentiation of a hES cell, the method comprising co-culturing the hES cell with a cell excreting at least one cardiomyocyte differentiation inducing factor or with an extracellular medium therefrom, under conditions that induce differentiation, cells and cell populations so produced, and uses of the cells.

### TECHNICAL FIELD

The present invention relates to human embryonic stem cells (hES) and their differentiation. hES cells can give rise to cardiomyocytes that are a better model for studying human physiology than murine embryonic stem (mES) cell-derived cardiomyocytes or primary cardiomyocytes from adult or fetal mice. hES cells can give rise to cardiomyocytes which provide a model of relevance to the study of human cardiac disease. hES cells can give rise to normal and mutant cardiomyocytes suitable for testing drugs.

### BACKGROUND ART

Cardiomyocytes have potential in restoring heart function after myocardial infarction or in heart failure. Human embryonic stem (hES) cells are a potential source of transplantable cardiomyocytes but detailed comparison of hES derived cardiomyocytes with primary human cardiomyocytes is necessary before transplantation into patients becomes feasible.

Ischemic heart disease is the leading cause of mortality in the western world. Oxygen deprivation and subsequent reperfusion initiates irreversible cell damage, eventually leading to widespread cell death and loss of function. Strategies to regenerate damaged cardiac tissue by cardiomyocyte transplantation may prevent or limit post-infarction cardiac failure. We have shown previously that visceral endoderm (VE) like cell lines induce undifferentiated mouse P19 embryonal carcinoma (EC) and mouse embryonic stem (mES) cells to aggregate spontaneously in co-culture and differentiate within a week to cultures containing beating muscle (1-3). This induction potential was specific for VE-like cells and also observed when aggregates of P19EC cells were grown in conditioned medium from one VE-like cell line, END-2. Moreover, Dyer et al (4) have shown that END-2 cells can induce the differentiation of epiblast cells from the mouse embryo to undergo hematopoiesis and vasculogenesis and respecify prospective neuroectodermal cell fate. This effect was largely attributable to Indian hedgehog (Ihh) a factor secreted by END-2 cells and VE of the mouse embryo.

Molecular pathways leading to specification and terminal differentiation of cardiomyocytes from embryonic mesoderm during development are still unclear. Data derived from chick and amphibian, suggested that cardiac progenitors require interaction with anterior endoderm and possibly the organizer for myocardial differentiation to take place (5-7). More recently, primitive streak and visceral embryonic endoderm were shown to be important for multistep induction through which cardiac progenitor cells acquired the competence to complete terminal differentiation at E7.5 of gestation in mice (8).

Zebrafish mutants lacking endoderm exhibit severe heart abnormalities (9) while ablation of endoderm in Xenopus laevis results in loss of expression of heart-specific cardiac troponin I (10) although expression of a general muscle marker is retained. The data indicate an important, even essential role for endoderm-mesoderm interaction in cardiomyocyte differentiation.

### DESCRIPTION OF THE INVENTION

Human embryonic stem cells co-cultured with mouse visceral endoderm (VE)-like cells form beating muscle cells, expressing cardiac specific sarcomeric proteins and ion channels. Direct comparison of electrophysiological responses demonstrated that the majority resembled human fetal ventricular cells in culture; a minority had an atrial phenotype. Both fetal and hES-derived cardiomyocytes in culture were functionally coupled through gap junctions. This is the first demonstration of induction of cardiomyocyte differentiation in hES cells that do not undergo cardiogenesis spontaneously.

Here, we demonstrate that co-culture of pluripotent hES cell lines with END-2 cells induces extensive differentiation to two distinctive cell types from different lineages. One is epithelial and forms large cystic structures staining positively for alpha-fetoprotein and is presumably extraembryonic visceral endoderm; the others are grouped in areas of high local density and beat spontaneously. We show that these beating cells are cardiomyocytes. Although differentiation of hES cells to cardiomyocytes has been described previously (11-13), the hES cell lines used differentiate spontaneously to somatic derivatives in embryoid bodies, reminiscent of those formed by mES cells (14). The present work is thus the first describing induction of cardiomyocyte differentiation in hES cells, which do not undergo cardiogenesis spontaneously, even at high local cell densities and is the first direct electrophysiological comparison of hES-derived cardiomyocytes with primary human fetal cardiomyocytes in culture.

In a first aspect the present invention provides a method for inducing cardiomyocyte differentiation of a human embryonic stem cell (hES ), the method comprising co-culturing the hES cell with a cell excreting at least one cardiomyocyte differentiation inducing factor or with an extracellular medium therefrom, under conditions that induce differentiation. Typically the cell produces a protein excretion profile that is at least substantially as produced by mouse VE-like cells.

The stem cells suitable for use in the present methods may be derived from a patient's own tissue. This would enhance compatibility of differentiated tissue grafts derived from the stem cells with the patient. In this context it should be noted that hES cells can include adult stem cells derived from a person's own tissue. Human stem cells may be genetically modified prior to use through introduction of genes that may control their state of differentiation prior to, during or after their exposure to the embryonic cell or extracellular medium from an embryonic cell. They may be genetically modified through introduction of vectors expressing a selectable marker under the control of a stem cell specific promoter such as Oct-4. The stem cells may be genetically modified at any stage with a marker so that the marker is carried through to any stage of cultivation. The marker may be used to purify the differentiated or undifferentiated stem cell populations at any stage of cultivation.

Cells providing differentiating factor(s) may be embryonic cells derived from visceral endoderm tissue or visceral endoderm like tissue isolated from an embryo. Preferably visceral endoderm may be isolated from early postgastrulation embryos, such as mouse embryo (E7.5). Visceral endoderm or visceral endoderm like tissue can be isolated as described in Roelen et al, 1994 Dev. Biol. 166:716-728. Characteristically the visceral endoderm may be identified by expression of alphafetoprotein and cytokeratin ENDO-A). The embryonic cell may be an embryonal carcinoma cell, preferably one that has visceral endoderm properties.

In one embodiment cell producing differentiation factor(s) is a mouse VE-like cell or a cell derived therefrom. In a preferred form of this embodiment the cell is an END-2 cell.

The embryonic stem cell may be derived from a cell line or cells in culture. The embryonic cell may be derived from an embryonic cell line, preferably a cell line with characteristics of visceral endoderm, such as the END-2 cell line (Mummery et al, 1985, Dev Biol. 109:402-410). The END-2 cell line was established by cloning from a culture of P19 EC cells treated as aggregates in suspension (embryoid bodies) with retinoic acid then replated (Mummery et al, 1985, Dev Biol. 109:402-410). The END-2 cell line has characteristics of visceral endoderm (VE), expressing alpha-fetoprotein (AFP) and the cytoskeletal protein ENDO-A.

In another embodiment the cell is a liver parenchymal cell. In a preferred form of this embodiment the liver parenchymal cell is HepG2.

The human embryonic stem cell may be derived directly from an embryo or from a culture of embryonic stem cells. (see for example Reubinoff et al., Nature Biotech. 16:399-404 2000). The stem cell may be derived from an embryonic cell line or embryonic tissue. The embryonic stem cells may be cells which have been cultured and maintained in an undifferentiated state.

The hES cells may be hES cells which do not undergo cardiogenesis spontaneously.

In a second aspect the present invention provides a differentiated cardiomyocyte produced from an hES cell that does not undergo cardiogenesis spontaneously.

The invention also provides a cardiomyocyte produced by a method according to the first aspect of the invention.

The differentiated cardiomyocyte may express cardiac specific sarcomeric proteins and display chronotropic responses and ion channel expression and function typical of cardiomyocytes.

Preferably, the differentiated cardiomyocyte resembles a human fetal ventricular cell in culture.

In another preferred form the differentiated cardiomyocyte resembles a human fetal atrial cell in culture.

In another preferred form the differentiated cardiomyocyte resembles a human fetal pacemaker cell in culture.

It will be understood that the resemblance to these fetal cells does not necessarily extend to possessing the same level of maturity as these fetal cells.

In a third aspect the present invention provides a plurality of differentiated cardiomyocytes of the invention wherein the differentiated cardiomyocytes are coupled. The coupling may be functional or physical.

In one embodiment the coupling is through gap junctions.

In another embodiment the coupling is through adherens junctions.

In a further embodiment the coupling is electrical.

In a fourth aspect the present invention provides a colony of differentiated cardiomyocytes produced by dissociating beating areas from differentiated cardiomyocytes of the invention.

Typically the dissociated cells are replated. Preferably they adopt a two dimensional morphology.

In a fifth aspect the present invention provides a model for the study of human cardiomyocytes in culture, comprising differentiated cardiomyocytes of the invention. This model is useful in the development of cardiomyocyte transplantation therapies.

In a sixth aspect the present invention provides an in vitro system for testing cardiovascular drugs comprising a differentiated cardiomyocyte of the invention.

In a seventh aspect the present invention provides a mutated differentiated cardiomyocyte of the invention prepared from a mutant hES cell. It will be recognized that methods for introducing mutations into cells are well known in the art.

In an eighth aspect the present invention provides a method of studying cardiomyocyte differentiation and function (electrophysiology) comprising use a mutated differentiated cardiomyocyte of the seventh aspect.

In a ninth aspect the present invention provides an in vitro system for testing cardiovascular drugs comprising a mutated differentiated cardiomyocyte of the seventh aspect.

In a tenth aspect the present invention provides an in vitro method for testing cardiovascular drugs comprising using a mutated differentiated cardiomyocyte of the seventh aspect as the test cell.

The present invention describes the genes and proteins present in cardiomyocytes derived from hES. Ion channels play an important role in cardiomyocyte function. If we know which channels are expressed we can make hES cells lacking specific ion channels, and study the effect on cardiac differentiation and function (using electrophysiology). Furthermore, drugs specific for a cardiac ion channel can be tested on cardiomyocyte function (looking at indicators such as action potential, beating frequency, and morphological appearance).

Expression of cardiac specific ion channels was determined in undifferentiated hES cells and in differentiating cells 8 and 15-days after initiation of co-culture with END-2 cells (FIG. 3). As shown by others previously (12), areas of beating hES-derived cardiomyocytes express ANF. Expression of the α-subunits of the cardiac specific L-type calcium channel (α1c) and the transient outward potassium channel (Kv4.3) were also detected, the expression of Kv4.3 preceding onset of beating by several days. RNA for the delayed rectifier potassium channel KvLQT1 was found in undifferentiated cells, but transcripts disappeared during early differentiation and reappeared at later stages.

Vital fluorescent staining with ryanodine or antibodies against cell surface α1c ion channels allowed differentiated cardiomyocytes of the invention to be identified in mixed cultures. This may provide a means of isolating cardiomyocytes for transplantation without genetic manipulation or compromising their viability.

The cells of the invention may be formulated with suitable carriers.

The present invention also provides differentiated cells produced according to the methods of the invention that may be used for transplantation, cell therapy or gene therapy. Preferably, the invention provides a differentiated cell produced according to the methods of the invention that may be used for therapeutic purposes, such as in methods of restoring cardiac function in a subject suffering from a heart disease or condition.

Another aspect of the invention is a method of treating or preventing a cardiac disease or condition, the method including introducing an isolated differentiated cardiomyocyte cell of the invention and/or a cell capable of differentiating into a cardiomyocyte cell when treated in accordance with the method of the first aspect of the invention into cardiac tissue of a subject. The isolated cardiomyocyte cell is preferably transplanted into damaged cardiac tissue of a subject. More preferably, the method results in the restoration of cardiac function in a subject.

In yet another aspect of the invention there is provided a method of repairing cardiac tissue, the method including

introducing an isolated cardiomyocyte cell of the invention and/or a cell capable of differentiating into a cardiomyocyte cell when treated in accordance with the method of the first aspect of the invention into damaged cardiac tissue of a subject.

It is preferred that the subject is suffering from a cardiac disease or condition. In the method of the present invention, the isolated cardiomyocyte cell is preferably transplanted into damaged cardiac tissue of a subject. More preferably, the method results in the restoration of cardiac function in a subject.

The present invention preferably also provides a myocardial model for testing the ability of stem cells that have differentiated into cardiomyocytes to restore cardiac function.

The present invention further provides a cell composition including a differentiated cell of the present invention, and a carrier.

The term “inducing differentiation” as used herein is taken to mean causing a stem cell to develop into a specific differentiated cell type as a result of a direct or intentional influence on the stem cell. Influencing factors can include cellular parameters such as ion influx, a pH change and/or extracellular factors, such as secreted proteins, such as but not limited to growth factors and cytokines that regulate and trigger differentiation. It may include culturing the cell to confluence and may be influenced by cell density.

Preferably, the hES cell and the cell providing the differentiating factor(s) are co-cultured in vitro. This typically involves introducing the stem cell to an embryonic cell monolayer produced by proliferation of the embryonic cell in culture. Preferably, the embryonic cell monolayer is grown to substantial confluence and the stem cell is allowed to grow in the presence of extracellular medium of the embryonic cells for a period of time sufficient to induce differentiation of the stem cell to a specific cell type. Alternatively, the stem cell may be allowed to grow in culture containing the extracellular medium of the embryonic cell(s), but not in the presence of the embryonic cell(s). The embryonic cells and stem cells may be separated from each other by a filter or an a cellular matrix such as agar.

In general for differentiation of stem cells the stem cell can be plated on a monolayer of embryonic cells and allowed to grow in culture to induce differentiation of the stem cell.

Conditions for obtaining differentiated embryonic stem cells are typically those which are non-permissive for stem cell renewal, but do not kill stem cells or drive them to differentiate exclusively into extraembryonic lineages. A gradual withdrawal from optimal conditions for stem cell growth favours differentiation of the stem cell to specific cell types. Suitable culture conditions may include the addition of DMSO, retinoic acid, FGFs or BMPs in co-culture which could increase differentiation rate and/or efficiency.

The cell density of the embryonic cell layer typically affects its stability and performance. The embryonic cells are typically confluent. Typically, the embryonic cells are grown to confluence and are then exposed to an agent which prevents further division of the cells, such as mitomycin C. The embryonic monolayer layer is typically established 2 days prior to addition of the stem cell(s). The stem cells are typically dispersed and then introduced to a monolayer of embryonic cells. Typically, the stem cells and embryonic cells are co-cultured for a period of two to three weeks until a substantial portion of the stem cells have differentiated.

The term “extracellular medium” as used herein is taken to mean conditioned medium produced from growing an embryonic cell as herein described in a medium for a period of time so that extracellular factors, such as secreted proteins, produced by the embryonic cell are present in the conditioned medium. The medium can include components that encourage the growth of the cells, for example basal medium such as Dulbecco's minimum essential medium, Ham's F12, or foetal calf serum.

The cardiomyocytes of the invention are preferably beating. Cardiomyocytes, can be fixed and stained with α-actinin antibodies to confirm muscle phenotype. α-troponin, α-tropomysin and α-MHC antibodies also give characteristic muscle staining. Preferably, the cardiomyocytes are fixed according to methods known to those skilled in the art. More preferably, the cardiomyocytes are fixed with paraformaldehyde, preferably with about 2% to about 4% paraformaldehyde. Ion channel characteristics and action potentials of muscle cells can be determined by patch clamp, electrophysiology and RT-PCR.

Stem cells from which cardiomyocytes are to be derived can be genetically modified to bear mutations in, for example, ion channels (this causes sudden death in humans). Cardiomyocytes derived from these modified stem cells will thus be abnormal and yield a culture model for cardiac ailments associated with defective ion channels. This would be useful for basic research and for testing pharmaceuticals. Likewise, models in culture for other genetically based cardiac diseases could be created. Cardiomyocytes of the present invention can also be used for transplantation and restoration of heart function.

For instance, ischaemic heart disease is the leading cause of morbidity and mortality in the western world. Cardiac ischaemia caused by oxygen deprivation and subsequent oxygen reperfusion initiates irreversible cell damage, eventually leading to widespread cell death and loss of function. Strategies to regenerate damaged cardiac tissue by cardiomyocyte transplantation may prevent or limit post-infarction cardiac failure. The methods of inducing stem cells to differentiate into cardiomyocytes, as hereinbefore described would be useful for treating such heart diseases. Cardiomyocytes of the invention may also be used in a myocardial infarction model for testing the ability to restore cardiac function.

The present invention preferably provides a myocardial model for testing the ability of stems cells that have differentiated into cardiomyocytes to restore cardiac function. In order to test the effectiveness of cardiomyocyte transplantation in vivo, it is important to have a reproducible animal model with a measurable parameter of cardiac function. The parameters used should clearly distinguish control and experimental animals (see for example Palmen et al. (2001), Cardiovasc. Res. 50, 516-524) so that the effects of transplantation can be adequately determined. PV relationships are a measure of the pumping capacity of the heart and may be used as a read-out of altered cardiac function following transplantation.

A host animal, such as but not limited to, an immunodeficient mouse may be used as a ‘universal acceptor’ of cardiomyocytes from various sources. The cardiomyocytes are produced by the method of the present invention.

The myocardial model of the present invention is preferably designed to assess the extent of cardiac repair following transplant of cardiomyocytes or suitable progenitors into a suitable host animal. More preferably, the host animal is an immunodeficient animal created as a model of cardiac muscle degeneration following infarct that is used as a universal acceptor of the differentiated cardiomyocytes. This animal can be any species including but not limited to murine, ovine, bovine, canine, porcine and any non-human primates. Parameters used to measure cardiac repair in these animals may include, but are not limited to, electrophysiological characteristic of heart tissue or various heart function. For instance, contractile function may be assessed in terms of volume and pressure changes in a heart. Preferably, ventricular contractile function is assessed. Methods of assessing heart function and cardiac tissue characteristics would involve techniques also known to those skilled in the field.

The present invention further provides a cell composition including a differentiated cell of the present invention, and a carrier. The carrier may be any physiologically acceptable carrier that maintains the cells. It may be PBS or other minimum essential medium known to those skilled in the field. The cell composition of the present invention can be used for biological analysis or medical purposes, such as transplantation.

The cell composition of the present invention can be used in methods of repairing or treating diseases or conditions, such as cardiac disease or where tissue damage has occurred. The treatment may include, but is not limited to, the administration of cells or cell compositions (either as partly or fully differentiated) into patients. These cells or cell compositions would result in reversal of the condition via the restoration of function as previously disclosed above through the use of animal models.

Throughout the description and claims of this specification, the word “comprise” and variations of the word, such as “comprising” and “comprises”, is not intended to exclude other additives, components, integers or steps. The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed in Australia before the priority date of each claim of this application.

The present invention will now be more fully described with reference to the accompanying examples and drawings. It should be understood, however that the description following is illustrative only and should not be taken in any way as a restriction on the generality of the invention described above.

### BRIEF DESCRIPTION OF THE ACCOMPANYING DRAWINGS

FIG. 1. Induction of differentiation of hES cells by co-culture with END-2 cells. A. Undifferentiated hES cell colony on MEF “feeder cells”. B. nuclear oct-4 staining of undifferentiated cells in area of colony indicated in A. C. hES cells after 11 days of coculture with END-2 cells with beating aggregate (arrow); D. Various morphologies of beating muscle aggregates. E. Phase-contrast image showing beating muscle areas in hES/END-2 co-culture. F. Phase-contrast image of a non-beating area with cardiomyocyte morphology. G. Dissociated hES aggregate, replated and beating as used for electophysiology. H. Cystic structures stained with alphafetoprotein.

FIG. 2. Cardiomyocyte markers in hES/END2 co-cultures compared with primary human fetal and adult cardiomyocytes. A-G,O. hES-derived cardiomyocytes. H-J,P. Human fetal ventricular cardiomyocytes. K,L. Human fetal atrial cardiomyocytes. M. Adult human ventricular cardiomyocytes. N. Adult human atrial cardiomyocytes. Cells were stained with Hoechst (A,C,M,N), anti-α actinin (green) (B,E,F,H,M,N), anti-MLC-2a (red) (N), anti-MLC-2v (red) (M) and anti-tropomyosin (green) (D,G,I, J,L). Vital staining of ryanodine receptors in hES derived cardiomyocytes (O) and human fetal ventricular cardiomyocytes (P).

FIG. 3. Expression of cardiomyocyte marker and ion channel mRNA in cocultures of hES and END-2 cells by RT-PCR. RT-PCR on hES cells co-cultured for 8d with END-2 cells (8d+END-2), hES beating muscle (BM), adult human heart and directly on RNA (-RT).

FIG. 4. Action potentials and chronotropic responses. A. Action potentials in hES derived beating cardiomyocytes and isolated human fetal ventricular and atrial cells. B. Effect of Verapamil on action potentials in hES-derived and primary human fetal cardiomyocytes (hfetal). C. Chronotropic responses of hES and human fetal cardiomyocytes to different stimuli; mean beat frequency ± S.E.M.

FIG. 5. Calcium transients and L-type calcium channels. A. The first image of an image stack (100 images, total time 30 seconds) of a group of 7 cells. The lines indicate line scans in time through the image stack. B. Intensity plot of the horizontal line through the image stack in time (time running from top to bottom). C. Intensity plot of the vertical line from the image (time running from left to right). D. Image stamps of the first 36 images, with time. E. Calcium transients in a single cell from the upper right corner of the image in A. The dots are the average value of the same region of interest in one cell in each slice in the image stack. F and G. Confocal images of a-actinin (green) and α1C (red) positive cells in hES (F) and human fetal ventricular cardiomyocytes (G).

FIG. 6. Junctional communication in hES-derived and human fetal cardiomyocytes. A, B. Human ventricular fetal cardiomyocytes. C, D. hES-derived cells. Double-staining of phalloidin (red) and anti-pan-cadherin (green) (A, C) or anti-Cx43 (green) (B, D). E. Injection of Lucifer yellow into a single cell (arrows) within a group of beating hES derived cardiomyocytes results in dye spreading to multiple cells (arrow heads, left bottom; phase contrast, right bottom) within minutes as determined by 2D projected Zseries (upper panel from left to right).

### BEST METHOD OF CARRYING OUT THE INVENTION

Summary of Methods and Results

hES cells were co-cultured with visceral-endoderm (VE) like cells from the mouse. This initiated differentiation to beating muscle. Sarcomeric marker proteins, chronotropic responses and ion channel expression and function were typical of cardiomyocytes. Electrophysiology demonstrated that most cells resembled human fetal ventricular cells, with atrial-like responses in a minority population. Real-time intracellular calcium measurements, lucifer yellow injection and connexin 43 expression demonstrated that fetal and hES derived cardiomyocytes are coupled by gap junctions in culture. Antibody staining and inhibition of electrical responses by Verapamil demonstrated the presence of functional α1c calcium ion channels.

### CONCLUSIONS

This is the first demonstration of induction of cardiomyocyte differentiation in hES cells that do not undergo spontaneous cardiogenesis. It provides a model for the study of human cardiomyocytes in culture and thus is useful in the development of cardiomyocyte transplantation therapies.

Methods

Cell Culture

END-2 cells and hES2 cells were cultured as described previously (1,15,16). To initiate cocultures, mitogenically inactive END-2 cell cultures, treated for 3 hr with mitomycin C (mit.C; 10 μg/ml) (1), replaced mouse embryonic fibroblasts (MEFs) as feeders for hES cells. Co-cultures were then grown for up to 6 weeks and scored for the presence of areas of beating muscle from 5 days onwards. HepG2 cells, a carcinoma cell line resembling liver parenchymal cells (17), were cultured in DMEM plus 10% fetal calf serum (FCS) and passaged twice weekly. Co-cultures were initiated as for END-2 cells. For electrophysiology, beating aggregates were dissociated using collagenase and replated on gelatine-coated coverslips.

Immunohistochemistry

Cells were fixed with 3.0% paraformaldehyde, then permeablized with 0.1% triton X 100. Undifferentiated hES colonies were then stained overnight at 4° C. with anti-oct4 (Sigma), visualized using the ABC complex/HPR kit (DAKO) and the Fast 3,3″-diaminobenzidine tablet set (Sigma). For immunofluorescence antibodies against α-actinin, tropomyosin and pan-cadherin (Sigma), MLC2a and 2v (gift of Dr K. Chien), α1C and Cav1.2a (Alomone labs, Israel), connexin 43 (Transduction Labs, USA) and phalloidin-Cy3 (Sigma) were used in combination with fluorescent conjugated secondary antibodies (Jackson Laboratories, U.S.A.). Confocal images (Leica Systems) were made (63× objective) from 2D projected Z-series.

Primary Human Adult and Fetal Cardiomyocytes.

Primary tissue was obtained during cardiac surgery or following abortion after individual permission using standard informed consent procedures and approval of the ethics committee of the University Medical Center, Utrecht. Adult cardiomyocytes were isolated and cultured, as reported previously (3). Fetal cardiomyocytes were isolated from fetal hearts perfused by Langendorff and cultured on glass coverslips. For (patch clamp) electrophysiology, cells were collected in Tyrode's buffer with low Ca²⁺ (18).

RT-PCR

RNA was isolated using Ultraspec (Biotecx Laboratories) and reverse transcribed (500 ng total RNA) as described previously₁₉. Primer sequences and conditions for PCR are given in Table 1. Products were analyzed on an ethidium bromide stained 1.5% agarose gel. β-actin or β-tubulin were used as controls for RNA input.

TABLE 1
PCR primers and PCR conditions.

| | | Product | Annealing |
|---|---|---|---|
| Gene | Primer (SEQ ID No:) | size | T |
| Oct-4 | 5′-GAGAACAATGAGAACCTTCAGGAGA (1) | 215 | 55 |
| | 5′-TTCTGGCGCCGGTTACAGAACCA (2) | | |
| α-actinin | 5′-GGCGTGCAGTACAACTACGTG (3) | 580 | 56 |
| | 5′-AGTCAATGAGGTCAGGCCGGT (4) | | |
| ANF | 5′-GAACCAGAGGGGAGAGACAGAG (5) | 406 | 61 |
| | 5′-CCCTCAGCTTGCTTTTTAGGAG (6) | | |
| MLC-2v | 5′-GCGCCAAC TCCAACGTGTTCT (7) | 444 | 55 |
| First round | 5′-GTGATGATGTGCACCAGGTTC (8) | | |
| Nested PCR | 5′-AGGAGGCCTTCACTATCATGG (9) | | 55 |
| | 5′-GTGATGATGTGCACCAGGTTC (10) | | |
| MLC-2a | 5′-GAGGAGAATGGCCAGCAGGAA (11) | 449 | 55 |
| | 5′-GCGAACATCTGCTCCACCTCA (12) | | |
| Phospholamban | 5′-ACAGCTGCCAAGGCTACCTA (13) | 191 | 55 |
| | 5′-GCTTTTGACGTGCTTGTTGA (14) | | |
| α1c | 5′-CTGGACAAGAACCAGCGACAGTGCG (15) | 562 | 56 |
| | 5′-ATCACGATCAGGAGGGCCACATAGGG (16) | | |
| Kv4.3 | 5′-CTGGACAAGAACCAGCGACAGTGCG (17) | 322 | 55 |
| | 5′-ATCACGATCAGGAGGGCCACATAGGG (18) | | |
| KvLQT1 | 5′-TTCTTGGCTCGGGGTTTGCC (19) | 723 | 58 |
| | 5′-TGTTGCTGCCGCGATCCTTG (20) | | |
| β-actin | 5′-CCTGAACCCTAAGGCCAACCG (21) | 400 | 55 |
| | 5′-GCTCATAGCTCTTCTCCAGGG (22) | | |
| β-tubulin | 5′-TGGCTTTGCCCCTCTCACCA (23) | 369 | 61 |
| | 5′-CGGCGGAACATGGCAGTGAA (24) | | |

Electrophysiology

Data were recorded from cells at 33° C. in spontaneously beating areas using an Axopatch 200B amplifier (Axon Instruments Inc., Foster City, Calif., U.S.A.). Cell attached patches were made in the whole cell voltage-clamp mode. The pipette offset, series resistance and transient cancellation were compensated; subsequent action potentials were recorded by switching to the current-clamp mode of the 200B amplifier. Output signals were digitized at 4 kHz using a Pentium III equipped with an AD/DAC LAB PC+ acquisition board (National Instruments, Austin, Tex., U.S.A.). Patch pipettes with a resistance between 1 and 3 MΩ were used. Bath medium was 140 mM NaCl, 5 mM KCL, 2 mM CaCl₂, 10 mM HEPES, adjusted to pH 7.45 with NaOH. Pipette composition: 145 mM KCl, 5 mM NaCl, 2 mM CaCl₂, 4 mM EGTA, 2 mM MgCl₂, 10 mM HEPES, adjusted to pH 7.30 with KOH. Verapamil was used at 5 μM, as indicated.

Calcium Measurements.

Cells were labelled for 15 min at 37° C. with 10 μM fura2-AM. The light from two excitation monochromators (SPEX fluorolog SPEX Industries EDISON, N.J., U.S.A.) was rapidly alternated between 340 (8) nm and 380 (8) nm and coupled into a microscope via a UV-optic fiber. Fluorescence intensity images were recorded from living cells at a maximal rate 120 ms/pair and corrected for background fluorescence. Calibration used the minimal ratio (Rₘᵢₙ) after addition of 5 μg/ml ionomycin and 4 mM EGTA (pH 8) to the cells and the maximal ratio (Rₘₐₓ), after addition of 5 μg/ml ionomycin and 10 mM CaCl₂. The calcium concentration was calculated as follows: (R−Rₘᵢₙ)/(Rₘₐₓ−R)*sf2/sb2*K_{d} (20).

Dye Coupling

A filtered solution of 3% w/v Lucifer yellow Lithium salt (Molecular Probes, Leiden, NL) in 150 mM LiCl was microinjected through Quickfill glass microelectrodes (Clark Electromedical Instruments Pangbourne, UK). Dye was injected into one of a group of spontaneously beating cells by a 1 Hz square pulse (50% duty cycle), amplitude of 5×10⁻⁹ A. Directly after injection confocal laser scanning microscope images were made of the injected areas.

Results

Cardiomyocyte Differentiation of Human ES Cells.

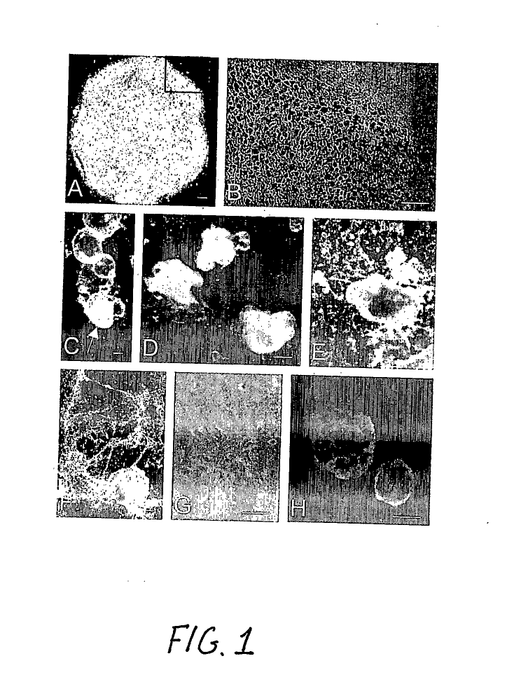

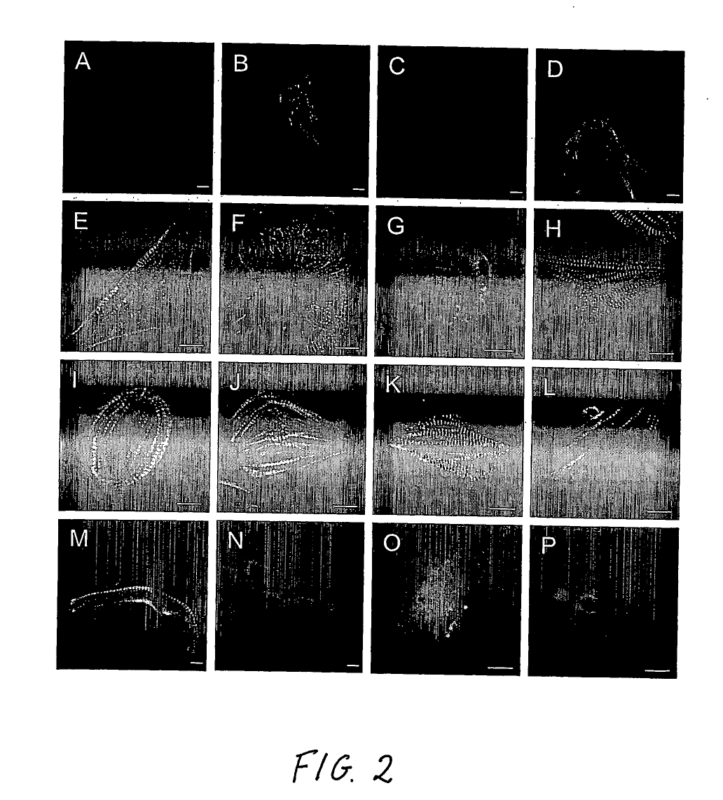

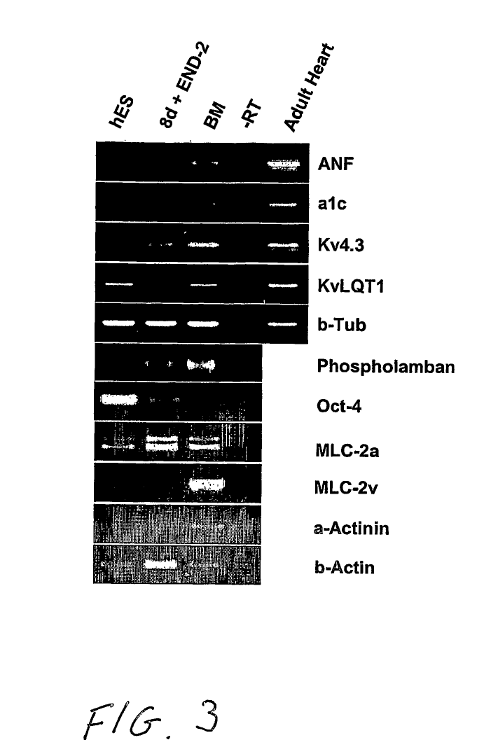

For undifferentiated hES cells maintained by co-culture with mit.C-treated MEFs in FCS containing medium (3) (FIG. 1A) ˜60% of cells showed nuclear staining for oct-4; any flattened cells were negative (FIG. 1B). Oct-4 expression thus correlated with phenotypic characteristics of undifferentiated cells. hES cells were subcultured by transferring small clumps of undifferentiated cells either to new MEFs or confluent cultures of END-2 cells. After approximately 5d, epithelial cells became evident which gradually become fluid-filled cysts (FIG. 1C). These stained for alphafetoprotein (FIG. 1H), suggesting they represent extraembryonic visceral endoderm. Control hES cells on MEFs were as shown in FIG. 1A. By 10d areas of rhythmically contracting cells in more solid aggregates became evident in the hES-END-2 co-cultures (arrow, FIG. 1C) with a variety of overall morphologies (FIG. 1D). 35±10% (n=30) of wells in a 12-well plate contained beating areas each of which could be dissociated and replated to yield up to 12 new colonies of beating cells with a 2-D rather than 3-D morphology (FIG. 1G); this facilitated access to the cells for characterization by patch-clamp electrophysiology. Control cultures on MEFs showed no evidence of beating muscle or extensive cyst formation but had formed very large colonies with many flattened cells at the colony edges (not shown). HES on HepG2 cells on the other hand did form areas of beating muscle as in FIGS. 1C and D, usually attached to HepG2 cell colonies. Before and after dissociation, hES-derived cardiomyocytes beat 35-90 times per minute (Table 2). Cardiomyocyte colonies could be frozen and sometimes resumed beating upon thawing. To characterize the cardiomyocytes further, we carried out immunofluorescent staining for sarcomeric proteins (FIG. 2A-G), using BIDOPY-ryanodine as a vital stain for ryanodine receptors in the sarcoplasmic reticulum (FIGS. 2O, P) and analyzed the expression of ion channels by RT-PCR (FIG. 3). In each case, we used primary human fetal (16-17 weeks) and adult atrial and/or ventricular tissue as controls. The data showed that hES-derived cardiomyocytes exhibited sarcomeric striations when stained with α actinin (FIGS. 2E, F), organized in separated bundles. These were reminiscent of the bundles observed in human fetal cardiomyocytes (FIGS. 2H, K), although the individual sarcomeres were less well defined. The morphology was quite different from the highly organized, parallel bundles observed in cells from biopsies of adult human heart (FIGS. 2M, N). hES-derived cardiomyocytes also stained with myosin light chain-2a, MLC-2v (data not shown) and tropomyosin (FIG. 2G) although again the sarcomeres were less evident than in human fetal and adult cardiomyocytes (FIGS. 2I, J).

Expression of Cardiac Ion Channels and Stem Cell-/Sarcomere Markers in hES/END-2 Co-cultures.

Expression of cardiac specific ion channels was determined in undifferentiated hES cells and in differentiating cells 8 and 15-days after initiation of co-culture with END-2 cells (FIG. 3). As shown by others previously (12), areas of beating hES-derived cardiomyocytes express ANF. Expression of the α-subunits of the cardiac specific L-type calcium channel (α1 c) and the transient outward potassium channel (Kv4.3) were also detected, the expression of Kv4.3 preceding onset of beating by several days. RNA for the delayed rectifier potassium channel KvLQT1 was found in undifferentiated cells, but transcripts disappeared during early differentiation and reappeared at later stages.

Over a similar time course, expression of oct-4 was reduced while transcripts for α actinin, MLC-2a and MLC-2v became detectable, reflecting the results of antibody staining.

Electrophysiology

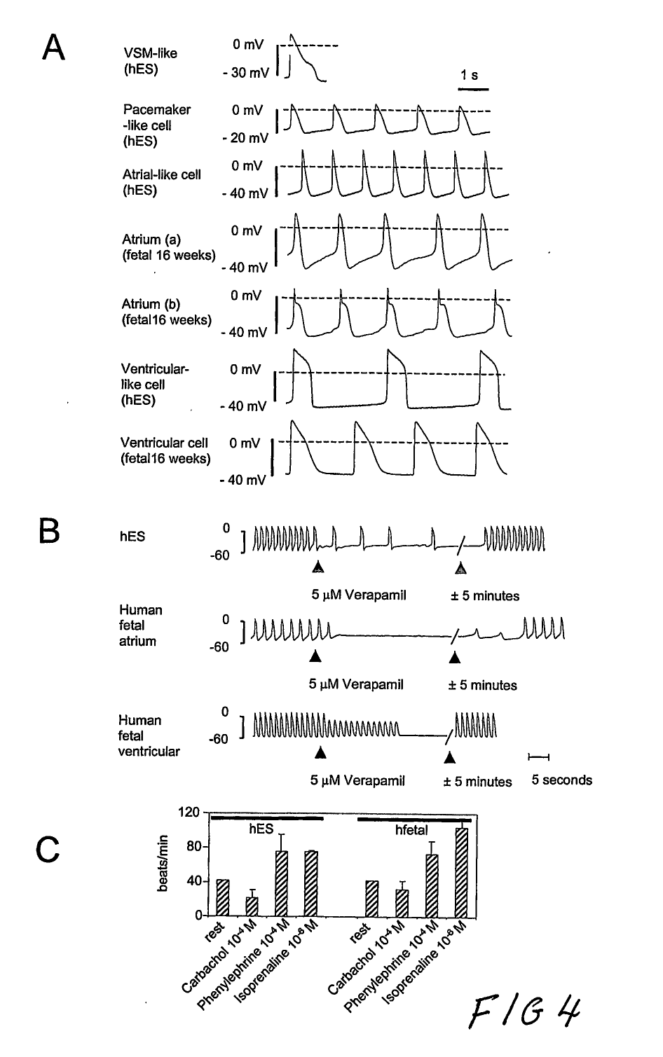

Patch clamp electrophysiology on dissociated hES cardiomyocytes showed that different (electrical) phenotypes were present (FIG. 4A). Ventricular-like action potentials predominated (28 out of 33; table 2) but atrial-like (n=2), pacemaker-like (n=1) and vascular smooth muscle like-cells (n=2) were also found. In areas in which the cells were not beating but had adopted morphologies indistinguishable from beating areas (FIG. 1F), current injection was sufficient to induce repeated action potentials and sustained synchronous rhythmic contractions. Transcripts for MLC-2v were also detected by RTPCR in non-beating, myocyte-like areas (not shown); scoring beating muscle may thus underestimate the number of cardiomyocytes present in culture. The upstroke velocities (V/s) for the ventricular-like cells were low (8 V/s) but comparable with those in cultured human fetal ventricular cardiomyocytes, although incidental peak values were found (Table 2). α1-adrenoceptors, β1-adrenoceptors (regulated via a cAMP-dependent mechanism) and nicotinic acetylcholine receptors are known to influence cardiac function. The effects of phenylephrine, isoprenaline and carbachol on cultured dissociated hES cardiomyocytes were tested and compared to cultured human fetal ventricular cells (FIG. 4B). Carbachol addition decreased the beating rate of hES derived cardiomyocytes and human fetal ventricular cells while an increase in response to phenylephrine and isoprenaline was observed in both cell types. Similar effects were reported in mES derived cardiomyocytes 21 and mouse fetal cells (22).

TABLE 2
Summary of cardiomyocyte action potential properties.

| | | Repo- | | Repo- | | | | Resting | | |
|---|---|---|---|---|---|---|---|---|---|---|
| | Upstroke | larisation | Repolarisation | larisation | Repolarisation | Overshoot | Amplitude | potential | Frequency | n |
| Cell type | V/s | 10% (mV) | 10% (ms) | 90% (mV) | 90% (ms) | (mV) | (mV) | (mV) | (Hz) | (total) |
| Pace maker | 2.6 | 4.8 | 72.0 | −20.8 | 134 | 18.0 | 32.0 | −20.8 | 1.2 | 1(33) |
| cells hES | | | | | | | | | | |
| (mean) | | | | | | | | | | |
| Atrium-like | 8.5 ± 0.4 | 9.9 ± 0.8 | 28.5 ± 11.0 | −38.7 ± 0.6 | 121.8 ± 3.8 | 26.0 ± 1.0 | 60.8 ± 3.2 | −38.7 ± 0.6 | 1.5 ± 0.1 | 2(33) |
| cells | | | | | | | | | | |
| hES | | | | | | | | | | |
| (mean ± SEM) | | | | | | | | | | |
| Atrium cells | 1.2 ± 0.3 | 10.9 ± 3.3 | 58.9 ± 10.0 | −34.9 ± 10 | 164.9 ± 14.3 | 16.6 ± 3.8 | 57.2 ± 5.0 | −34.9 ± 1.6 | 1.0 ± 0.1 | (8) |
| Foetal 16 | | | | | | | | | | |
| weeks | | | | | | | | | | |
| (mean ± SEM) | | | | | | | | | | |
| Ventricular- | 7.0 ± 0.8* | 16.0 ± 1.6 | 123.9 ± 19.5 | −48.0 ± 1.7 | 436.4 ± 55.3 | 24.0 ± 1.9 | 80.0 ± 3.5 | −48.0 ± 1.7 | 0.6 ± 0.1 | 28(33) |
| like cells | | | | | | | | | | |
| hES | | | | | | | | | | |
| (mean ± SEM) | | | | | | | | | | |
| Ventricular | 8.9 ± 4.3** | 16.7 ± 2.7 | 80.2 ± 13.2 | −38.5 ± 1.6 | 370.0 ± 45.8 | 23.6 ± 3.5 | 69.0 ± 9.1 | −38.5 ± 1.6 | 0.8 ± 0.1 | (6) |
| cells | | | | | | | | | | |
| Foetal 16 | | | | | | | | | | |
| weeks | | | | | | | | | | |
| (mean ± SEM) | | | | | | | | | | |

*Measured maximum: 20.0 (V/s)
**Measured maximum: 26.7 (V/s)

Measured mean values with standard error of the mean (S.E.M.) are indicated. The upstroke velocities (V/s) were measured with recordings maximally expanded in time, in the linear part of the upstroke trajectory.

[Ca²⁺]ᵢ Transients in Differentiated hES Cells.

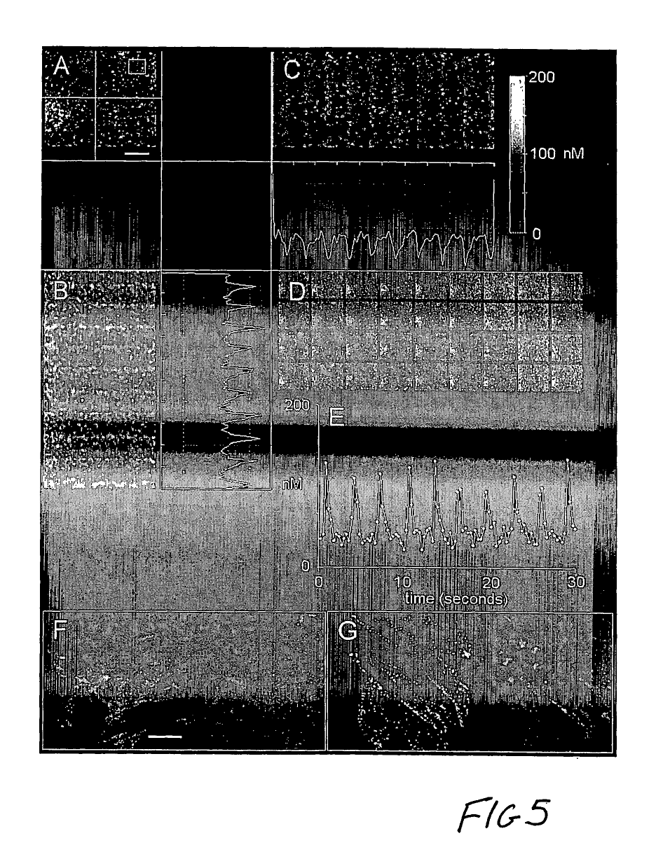

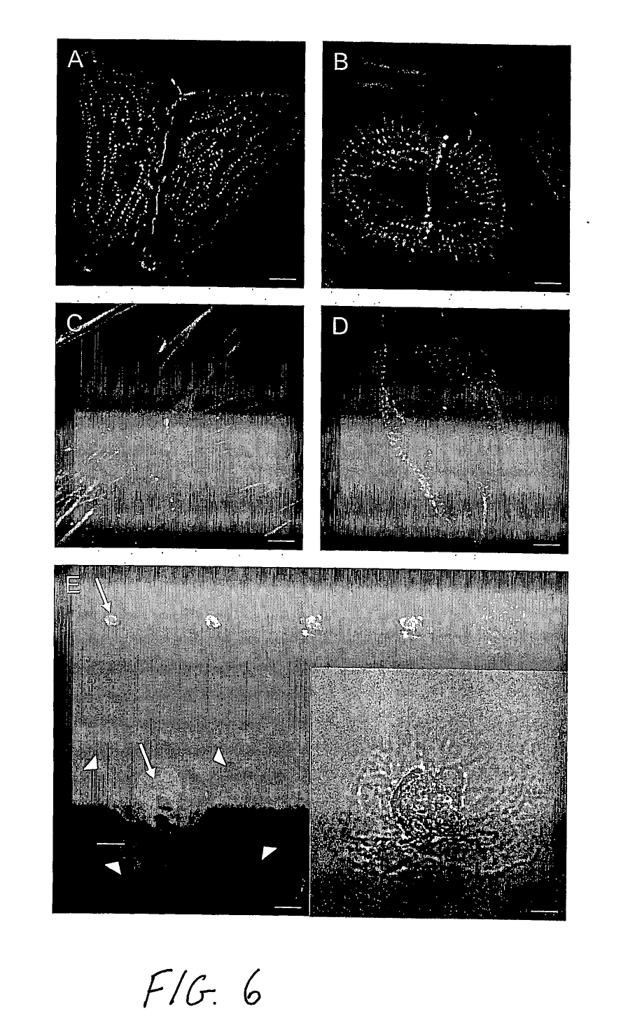

Calcium oscillations were recorded in dissociated groups of spontaneously beating hES cardiomyocytes (FIG. 5). The continuous character of the repetitive line scans in FIG. 5B in the left-right direction compared to the vertical lines in 5C, shows that the action potential in FIG. 4A propagates in a top down direction. This continuous aspect is also indicative of tightly developed cell-cell coupling in this synchronously contracting group of cells. Regular repetitive oscillations in [Ca²⁺]ᵢ are found in a single hES cardiomyocyte (FIG. 5E). Coupling between cells was confirmed by Lucifer yellow injection into single cells, which showed spreading of the dye within minutes to other cells within the group both in hES-derived (FIG. 6E) and primary fetal cardiomyocytes (not shown) and the presence of Cx43 staining between individual cells (FIGS. 6B, D) indicating the presence of gap junctions. Staining with a pan-cadherin antibody also indicated the presence of adherens junctions between cells in groups of fetal primary cardiomyocytes and hES-derived cells (FIGS. 6A, C).

L-type calcium channels comprise the predominant route for calcium entry into cardiac myocytes and are key components in excitation contraction coupling. The dominant cardiac specific isoform is α1 C (23). Using a specific α1 C antibody (24) we observed positive cardiomyocytes in both differentiated hES cultures (FIG. 5F) and in human fetal ventricular cells (FIG. 5G). This is in agreement with the RT-PCR data (FIG. 2)

Discussion

Before hES cells can be applied clinically it is important to control their growth and differentiation. Both embryonic and adult stem cells from the mouse apparently respond to cues within the mouse embryo to differentiate to (virtually) all somatic tissues (reviewed in 25). If these cues and the signal transduction pathways they activate can be identified, this knowledge can be utilized in controlling differentiation of stem cells in culture and in vivo. Here, we have identified (visceral-) endoderm as a cellular source of signals that result in human ES cells differentiating to cardiomyocytes with characteristics of fetal ventricular, atrial or pacemaker cells. This is the first time that inductive cellular sources of signals have been identified that result in human ES cells forming cardiomyocytes, although various studies have shown that cells with endodermlike properties have this effect on mouse ES and EC cells (1, 3, 26-28). VE (END-2) and liver parenchymal (HepG2) cells share similar protein secretion profiles so their ability to induce comparable responses in ES cells is not surprising. In contrast to mouse ES cells, in our hands human ES cells do not easily form embryoid bodies when grown as aggregates, and never show “spontaneous” differentiation to cardiomyocytes even at high cell densities in overgrowths. This contrasts with other reports (11-13) where the hES cells do form embryoid bodies containing cardiomyocytes. Identification of a reproducible source of inductive signals nevertheless represents an important step forward, comparable to a recent report showing differentiation of hES cells to haematopoietic cells after coculture with a bone marrow stromal cells or yolk sac endothelial cells (29). Among the cardiogenic signals emanating from endoderm that could be responsible for the effects in tissue recombination experiments in Xenopus and chick and in mutant zebrafish, described above, it has been suggested that BMPs, FGFs and repressors of wnt signaling may be the most important (reviewed Olson 30). Endoderm in the mouse embryo expresses BMP2 (31) and inhibitors of wnt signaling (32, 33). Direct addition of BMP2 to hES cells however, did not result in cardiomyocyte differentiation; on the contrary, they appeared to form extraembryonic endoderm (data not shown). We therefore think it unlikely that activation of the BMP signaling pathway is the primary event initiated by END-2/hES cell co-culture. Likewise, we saw no obvious effect of FGFs. These signals could however, be involved later in differentiation of nascent mesoderm to cardiomyoblasts and use of BMPs, FGFs and wnt antagonists to enhance differentiation described here is noted as possible. Late addition of the demethylating agent 5-azacytidine to developing embryoid bodies has also been shown to be more effective than early addition (13). Careful stepwise analysis of hES cell differentiation and approaches recapitulating or mimicking endogenous signals in the embryo are the most likely to increase the efficiencies of hES differentiation to specific lineages. In addition, transplantation of committed but immature cells that have retained the capacity to form functional junctions with host cells are likely to have the least chance of introducing arrythmias.

Sarcomere organization is largely determined by mechanical forces, which are relatively minor in culture compared with the intact heart. This may explain the poorly defined staining observed here which did not change over 6 weeks despite maintenance of beating (not shown). Likewise, staining for junctional proteins showed that the hES derived cardiomyocytes were very immature although real time determination of intracellular Ca²⁺ concentrations clearly showed that the cells were electrically coupled.

Kehat et al (11) recently reported similar findings in independently derived hES cardiomyocytes.

In the adult mammalian myocardium, cellular Ca²⁺ entry is regulated by the sympathetic nervous system. L-type Ca²⁺ channel currents are markedly increased by beta-adrenergic (beta-A) agonists, which contribute to changes in rate and contractile activity of the heart.

In the developing mammalian heart, the regulation of Ca²⁺ entry by this enzyme cascade has not been clearly established, because changes in receptor density and coupling to downstream elements of the signaling cascade occur as development proceeds. Our data indicate that the L-type Ca²⁺ channels in hES-derived cardiomyocytes and fetal cardiomyocytes responded to adrenergic stimuli, indicating a fully developed and connected downstream pathway. Verapamil, which specifically blocks L-type Ca²⁺ channels, inhibited action potentials in fetal and hES-derived cardiomyocytes as expected. This contrasts with mouse fetal myocytes and mES derived cardiomyocytes where early cells were non-responsive despite the presence of L-type Ca²⁺ channels. Here, the lack of cAMP-dependent protein kinase appeared to be the limiting factor (14, 22). Thus although hES-derived and early human fetal cardiomyocytes show some features of early mouse cardiomyocytes, their calcium channel modulation resembles that in the adult mouse. hES cells may thus represent an excellent system for studying changes in calcium channel function during early human development which appears to differ significantly from that in mice. Furthermore, the appropriate calcium handling makes the cells more suitable for transplantation. Interesting was the observation of cells with plateau and nonplateau type action potentials in the fetal atrial cultures. These have been described dispersed throughout the atrium of intact fetal hearts (34) and have been considered as a possible index of specialization of an atrial fibre, although their significance is not clear. The nonplateau type was not observed among the hES-derived cardiomyocytes.

Finally, we showed that vital fluorescent staining with ryanodine or antibodies against cell surface α1c ion channels allowed these cells to be identified in mixed cultures. This may provide a means of isolating cardiomyocytes for transplantation without genetic manipulation or compromising their viability.

### REFERENCES

1. Mummery C L, van Achterberg T A, van den Eijnden-van Raaij A J et al. Visceral endoderm-like cell lines induce differentiation of murine P19 embryonal carcinoma cells. Differentiation. 1991; 46:51-60.
2. Rohwedel J, Maltsev V, Bober E et al. Muscle cell differentiation of embryonic stem cells reflects myogenesis in vivo: developmentally regulated expression of myogenic determination genes and functional expression of ionic currents. Dev Biol. 1994; 164:87-101.
3. Mummery C, Ward D, van den Brink C E et al. Cardiomyocyte differentiation of mouse and human embryonic stem cells. J Anat 2002; 200:233-242.
4. Dyer M A, Farrington S M, Mohn D et al. Indian hedgehog activates hematopoiesis and vasculogenesis and can respecify prospective neurectodermal cell fate in the mouse embryo. Development 2001; 128:1717-1730.
5. Garcia-Martinez V, Schoenwolf G C. Primitive-streak origin of the cardiovascular system in avian embryos. Dev Biol. 1993; 159:706-719.
6. Sugi Y, Lough J. Anterior endoderm is a specific effector of terminal cardiac myocyte differentiation of cells from the embryonic heart forming region. Dev Dyn. 1994; 200: 155-162.
7. Nascone N, Mercola M. An inductive role for the endoderm in Xenopus cardiogenesis. Development. 1995; 121:515-523.
8. Arai A, Yamamoto K, Toyama J. Murine cardiac progenitor cells require visceral embryonic endoderm and primitive streak for terminal differentiation. Dev Dyn. 1997; 210:344-353.
9. Kikuchi Y, Agathon A, Alexander J et al. casanova encodes a novel Sox-related protein necessary and sufficient for early endoderm formation in zebrafish. Genes Dev 2001; 15:1493-1505.
10. Vokes S A, Krieg P A. Endoderm is required for vascular endothelial tube formation, but not for angioblast specification. Development 2002; 129:775-785.
11. Kehat I, Gepstein A, Spira A et al. High-resolution electrophysiological assessment of human embryonic stem cell-derived cardiomyocytes: a novel in vitro model for the study of conduction. Circ Res 2002; 91:659-661.
12. Kehat I, Kenyagin-Karsenti D, Snir M et al. Human embryonic stem cells can differentiate into myocytes with structural and functional properties of cardiomyocytes. J Clin Invest 2001; 108:407-414.
13. Xu C, Inokuma M S, Denham J et al. Feeder-free growth of undifferentiated human embryonic stem cells. Nat Biotechnol 2001; 19:971-974.
14. Doevendans P A, Kubalak S W, An R H et al. Differentiation of cardiomyocytes in floating embryoid bodies is comparable to fetal cardiomyocytes. J Mol Cell Cardiol 2000; 32:839-851.
15. van den Eijnden-van Raaij A J, van Achterberg T A, van der Kruijssen C M et al. Differentiation of aggregated murine P19 embryonal carcinoma cells is induced by a novel visceral endoderm-specific FGF-like factor and inhibited by activin A. Mech Dev. 1991; 33:157-165.
16. Reubinoff B E, Pera M F, Fong C Y et al. Embryonic stem cell lines from human blastocysts: somatic differentiation in vitro. Nat Biotechnol 2000; 18:399-404.
17. Knowles B B, Howe C C, Aden D P. Human hepatocellular carcinoma cell lines secrete the major plasma proteins and hepatitis B surface antigen. Science. 1980; 209:497-499.
18. Sipido K R, Maes M, Van de Werf F. Low efficiency of Ca2+ entry through the Na(+)-Ca2+ exchanger as trigger for Ca2+ release from the sarcoplasmic reticulum. A comparison between L-type Ca2+ current and reverse-mode Na(+)-Ca2+ exchange. Circ Res. 1997; 81:1034-1044.
19. Goumans M J, Zwijsen A, van Rooijen M A et al. Transforming growth factor-beta signalling in extraembryonic mesoderm is required for yolk sac vasculogenesis in mice. Development. 1999; 126:3473-3483.
20. Grynkiewicz G, Poenie M, Tsien R Y. A new generation of Ca2+ indicators with greatly improved fluorescence properties. J Biol Chem. 1985; 260:3440-3450.
21. Wobus A M, Wallukat G, Hescheler J. Pluripotent mouse embryonic stem cells are able to differentiate into cardiomyocytes expressing chronotropic responses to adrenergic and cholinergic agents and Ca2+ channel blockers. Differentiation. 1991; 48:173-182.
22. An R H, Davies M P, Doevendans P A et al. Developmental changes in betaadrenergic modulation of L-type Ca2+ channels in embryonic mouse heart. Circ Res. 1996; 78:371-378.
23. Striessnig J. Pharmacology, structure and function of cardiac L-type Ca(2+) channels. Cell Physiol Biochem. 1999; 9:242-269.
24. Liu R, Li W. Karin N J et al. Ribozyme ablation demonstrates that the cardiac subtype of the voltage-sensitive calcium channel is the molecular transducer of 1,25-dihydroxyvitamin D(3)-stimulated calcium influx in osteoblastic cells. J Biol Chem 2000; 275:8711-8718.
25. Passier R., Mummery C. L. The origin and use of embryonic and adult stem cells in differentiation and tissue repair. Card Res. In press.
26. Rathjen J, Haines B P, Hudson K M et al. Directed differentiation of pluripotent cells to neural lineages: homogeneous formation and differentiation of a neurectoderm population. Development 2002; 129:2649-2661.
27. Rathjen J, Lake J A, Bettess M D et al. Formation of a primitive ectoderm like cell population, EPL cells, from ES cells in response to biologically derived factors. J Cell Sci. 1999; 112:601-612.
28. Lake J, Rathjen J, Remiszewski J et al. Reversible programming of pluripotent cell differentiation. J Cell Sci 2000; 113:555-566.
29. Kaufman D S, Hanson E T, Lewis R L et al. Hematopoietic colony-forming cells derived from human embryonic stem cells. Proc Natl Acad Sci USA 2001; 98:10716-10721.
30. Olson E N. Development. The path to the heart and the road not taken. Science 2001; 291 :2327-2328.
31. Coucouvanis E, Martin G R. Signals for death and survival: a two-step mechanism for cavitation in the vertebrate embryo. Cell. 1995; 83:279-287.
32. Piccolo S, Agius E, Leyns L et al. The head inducer Cerberus is a multifunctional antagonist of Nodal, BMP and Wnt signals. Nature. 1999; 397:707-710.
33. Mukhopadhyay M, Shtrom S, Rodriguez-Esteban C et al. Dickkopfl is required for embryonic head induction and limb morphogenesis in the mouse. Dev Cell 2001; 423-434.
34. Janse M K, Anderson R H, van Capelle F J et al. A combined electrophysiological and anatomical study of the human fetal heart. Am Heart J. 1976; 91:556-562.

1. A method for inducing cardiomyocyte differentiation of a human embryonic stem (hES) cell, the method comprising co-culturing the hES cell with a cell excreting at least one cardiomyocyte differentiation inducing factor or with an extracellular medium therefrom, under conditions that induce differentiation.

2. A method according to claim 1, wherein the cell excreting at least one cardiomyocyte differentiation inducing factor produces a protein excretion profile that is at least substantially as produced by mouse VE-like cells.

3. A method according to claim 1, wherein the hES cell is derived from a patient's own tissue.

4. A method according to claim 1, wherein the hES cell is genetically modified prior to use through introduction of genes that control the state of differentiation prior to, during or after their exposure to the cell excreting at least one cardiomyocyte differentiation inducing factor embryonic cell or extracellular medium therefrom.

5. A method according to claim 4, wherein the hES cell is genetically modified through introduction of a vector expressing a selectable marker under the control of a stem cell specific promoter.

6. A method according to claim 5, wherein the stem cell specific promoter is Oct-4.

7. A method according to claim 4, wherein the hES cell is genetically modified with a marker so that the marker is carried through to cultivation.

8. A method according to claim 7, wherein the marker is used to purify differentiated or undifferentiated hES cell populations during cultivation.

9. A method according to claim 1, wherein the cell excreting at least one cardiomyocyte differentiation inducing factor is an embryonic cell derived from visceral endoderm tissue or visceral endoderm like tissue isolated from an embryo.

10. A method according to claim 9, wherein the visceral endoderm tissue is isolated from an early postgastrulation embryo.

11. A method according to claim 10, wherein the early postgastrulation embryo is mouse embryo (E7.5).

12. A method according to claim 9, wherein the embryonic cell is an embryonal carcinoma cell.

13. A method according to claim 12, wherein the embryonal carcinoma cell has visceral endoderm properties.

14. A method according to claim 9, wherein the cell excreting at least one cardiomyocyte differentiation inducing factor is a mouse VE-like cell or a cell derived therefrom.

15. A method according to claim 14, wherein the cell is an END-2 cell.

16. A method according to claim 9, wherein the embryonic cell is derived from a cell line or lines in culture.

17. A method according to claim 16, wherein the embryonic cell is derived from an embryonic cell line.

18. A method according to claim 16, wherein the embryonic cell line is a cell line with characteristics of visceral endoderm.

19. A method according to claim 18, wherein the embryonic cell line is the END-2 cell line.

20. A method according to claim 1, wherein the cell excreting at least one cardiomyocyte differentiation inducing factor is a liver parenchymal cell.

21. A method according to claim 20, wherein the liver parenchymal cell is HepG2.

22. A method according to claim 1, wherein the hES cell is derived directly from an embryo or from a culture of embryonic stem cells.

23. A method according to claim 22, wherein the hES cell is derived from an embryonic cell line or embryonic tissue.

24. A method according to claim 23, wherein the hES cell has been cultured and maintained in an undifferentiated state.

25. A method according to claim 1, wherein the hES cell does not undergo cardiogenesis spontaneously.

26. A method according to claim 1, wherein the cardiomyocyte differentiation inducing factor is a secreted protein.

27. A method according to claim 26, wherein the secreted protein is a growth factor or cytokine that regulates and triggers differentiation.

28. A method according to claim 1, wherein the hES cell and the cell providing the differentiating factor(s) are co-cultured in vitro.

29. A method according to claim 28, which includes introducing hES cell to an embryonic cell monolayer produced by proliferation of the embryonic cell in culture.

30. A method according to claim 29, wherein the embryonic cell monolayer is grown to substantial confluence and the hES cell is allowed to grow in the presence of extracellular medium of the embryonic cells for a period of time sufficient to induce differentiation of the hES cell to a specific cell type.

31. A method according to claim 30, wherein the hES cell is allowed to grow in culture containing the extracellular medium of the embryonic cell(s), but not in the presence of the embryonic cell(s).

32. A method according to claim 31, wherein the embryonic cells and hES cells are separated from each other by a filter or an cellular matrix such as agar.

33. A method according to claim 1, wherein conditions for obtaining differentiated hES cells are those which are non-permissive for stem cell renewal, but do not kill stem cells or drive them to differentiate exclusively into extraembryonic lineages.

34. A method according to claim 33, wherein a gradual withdrawal from optimal conditions for hES cell growth favours differentiation of the hES cell to specific cell types.

35. A method according to claim 34, wherein suitable culture conditions include the addition of DMSO, retinoic acid, FGFs or BMP's in c-culture which could increase differentiation rate and/or efficiency.

36. A method according to claim 30, wherein the embryonic cells are grown to confluence and are then exposed to an agent which prevents further division of the cells.

37. A method according to claim 36, wherein the agent is mitomycin C.

38. A method according to claim 36, wherein the embryonic monolayer layer is established 2 days prior to addition of the hES cell(s).

39. A method according to claim 38, wherein the hES cells are dispersed and then introduced to a monolayer of embryonic cells.

40. A method according to claim 39, wherein the hES cells and embryonic cells are co-cultured for a period of two to three weeks until a substantial portion of the hES cells have differentiated.

41. A differentiated cardiomyocyte produced from an hES cell that does not undergo cardiogenesis spontaneously.

42. A differentiated cardiomyocyte produced by a method according to claim 1.

43. A differentiated cardiomyocyte according to claim 41, wherein the differentiated cardiomyocyte expresses cardiac specific sarcomeric proteins and displays chronotropic responses, ion channel expression and function typical of cardiomyocytes.

44. A differentiated cardiomyocyte according to claim 41, wherein the differentiated cardiomyocyte resembles a human fetal ventricular cell in culture.

45. A differentiated cardiomyocyte according to claim 41, wherein the differentiated cardiomyocyte resembles a human fetal atrial cell in culture.

46. A differentiated cardiomyocyte according to claim 41, wherein the differentiated cardiomyocyte resembles a human fetal pacemaker cell in culture.

47. A plurality of differentiated cardiomyocyte according to claim 41, wherein the differentiated cardiomyocytes are coupled.

48. A plurality of differentiated cardiomyocytes according to claim 47, wherein the coupling is functional.

49. A plurality of differentiated cardiomyocytes according to claim 47, wherein the coupling is physical.

50. A plurality of differentiated cardiomyocytes according to claim 47, wherein the coupling is through gap junctions.

51. A plurality of differentiated cardiomyocytes according to claim 47, wherein the coupling is through adherens junctions.

52. A plurality of differentiated cardiomyocytes according to claim 47, wherein the coupling is electrical.

53. A colony of differentiated cardiomyocytes according to claim 41.

54. A colony of differentiated cardiomyocytes produced by dissociating beating areas from differentiated cardiomyocytes according to claim 41.

55. A colony of differentiated cardiomyocytes produces by dissociating beating areas from differentiated cardiomyocytes produced by a method according to claim 1.

56. A colony according to claim 53, wherein the dissociated cells are replated.

57. A colony according to claim 56, wherein the dissociated cells adopt a two dimensional morphology.

58. A model for the study of human cardiomyocytes in culture, comprising differentiated cardiomyocytes according to claim 41.

59-65. (canceled)

66. An in vitro system for cardiovascular drug testing comprising a differentiated cardiomyocyte according to claim 41.

67. A mutated differentiated cardiomyocyte according to claim 41, performed on a mutant hES cell.

68. A method of studying cardiomyocyte differentiation and electrophysiology comprising use a mutated differentiated cardiomyocyte according to claim 67.

69. An in vitro system for cardiovascular drug testing comprising a mutated differentiated cardiomyocyte according to claim 67.

70. An in vitro method for cardiovascular drug testing comprising using a mutated differentiated cardiomyocyte according to claim 67 as the test cell.

71-72. (canceled)

73. A differentiated cardiomyocyte according to claim 41, formulated with a suitable carrier.

74-75. (canceled)

76. A method of treating or preventing a cardiac disease or condition, the method including introducing an isolated differentiated cardiomyocyte and/or a cell capable of differentiating into a cardiomyocyte cell when treated in accordance with a method according to claim 1 into cardiac tissue of a subject.

77. A method according to claim 76, wherein the isolated cardiomyocyte is transplanted into damaged cardiac tissue of a subject.

78. A method according to claim 77, wherein the method results in restoration of cardiac function in a subject.

79. A method of repairing cardiac tissue, the method including introducing an isolated differentiated cardiomyocyte produced from an hES cell that does not undergo cardiogenesis spontaneously and/or a cell capable of differentiating into a cardiomyocyte cell when treated in accordance with a method according to claim 1 into damaged cardiac tissue of a subject.

80. A method according to claim 79, wherein the subject is suffering from a cardiac disease or condition.

81. A method according to claim 79, wherein the method results in restoration of cardiac function in a subject.

82. A cell composition including a differentiated cardiomyocyte according to claim 41, and a carrier.

83-84. (canceled)

85. A myocardial model designed to assess the extent of cardiac repair following transplant wherein the host animal is an immunodeficient animal created as a model of cardiac muscles degeneration following infarct that is used as a universal acceptor of the differentiated cardiomyocyte according to claim 41.

86. A myocardial model according to claim 85, wherein the animal is murine, ovine, bovine, canine, porcine or a non-human primate.

87. A myocardial model according to claim 86, wherein electrophysiological characteristics of heart tissue or heart function is used to measure cardiac repair in these animals.

88. A myocardial model according to claim 87, wherein contractile function is assessed in terms of volume and pressure changes in a heart.

89. A myocardial model according to claim 87, wherein ventricular contractile function is assessed.

90. A differentiated cardiomyocyte according to claim 42, wherein the differentiated cardiomyocyte expresses cardiac specific sarcomeric proteins and displays chronotropic responses, ion channel expression and function typical of cardiomyocytes.

91. A differentiated cardiomyocyte according to claim 42, wherein the differentiated cardiomyocyte resembles a human fetal ventricular cell in culture.

92. A differentiated cardiomyocyte according to claim 42, wherein the differentiated cardiomyocyte resembles a human fetal atrial cell in culture.

93. A differentiated cardiomyocyte according to claim 42, wherein the differentiated cardiomyocyte resembles a human fetal pacemaker cell in culture.
